SEQUENCE LISTING (354 sequences)

SEQ ID NO: 1 - protein, 29 aa, Unknown
  polypeptide corresponding to a section of PrP27-30
GGGWGQGGGTHGQWNKPAKPKTAMKHXAG

SEQ ID NO: 2 - protein, 28 aa, Unknown
  polypeptide corresponding to a section of PrP27-30
GGWGQGGGTHGQWNKPAKPKTAMKHXAG

SEQ ID NO: 3 - protein, 27 aa, Unknown
  polypeptide corresponding to a section of PrP27-30
GWGQGGGTHGQWNKPAKPKTAMKHXAG

SEQ ID NO: 4 - protein, 26 aa, Unknown
  polypeptide corresponding to a section of PrP27-30
WGQGGGTHGQWNKPAKPKTAMKHXAG

SEQ ID NO: 5 - protein, 28 aa, Unknown
  polypeptide corresponding to a section of PrP27-30
GGGGQGGGTHGQWNKPAKPKTAMKHXAG

SEQ ID NO: 6 - protein, 27 aa, Unknown
  polypeptide corresponding to a section of PrP27-30
GGGQGGGTHGQWNKPAKPKTAMKHXAG

SEQ ID NO: 7 - protein, 26 aa, Unknown
  polypeptide corresponding to a section of PrP27-30
GGQGGGTHGQWNKPAKPKTAMKHXAG

SEQ ID NO: 8 - protein, 25 aa, Unknown
  polypeptide corresponding to a section of PrP27-30
GQGGGTHGQWNKPAKPKTAMKHXAG

SEQ ID NO: 9 - protein, 28 aa, Unknown
  polypeptide corresponding to a section of PrP27-30
GGGWGQGGGTHGQWNKPAKPKTAMKHXA

SEQ ID NO: 10 - protein, 27 aa, Unknown
  polypeptide corresponding to a section of PrP27-30
GGWGQGGGTHGQWNKPAKPKTAMKHXA

SEQ ID NO: 11 - protein, 26 aa, Unknown
  polypeptide corresponding to a section of PrP27-30
GWGQGGGTHGQWNKPAKPKTAMKHXA

SEQ ID NO: 12 - protein, 25 aa, Unknown
  polypeptide corresponding to a section of PrP27-30
WGQGGGTHGQWNKPAKPKTAMKHXA

SEQ ID NO: 13 - protein, 27 aa, Unknown
  polypeptide corresponding to a section of PrP27-30
GGGGQGGGTHGQWNKPAKPKTAMKHXA

SEQ ID NO: 14 - protein, 26 aa, Unknown
  polypeptide corresponding to a section of PrP27-30
GGGQGGGTHGQWNKPAKPKTAMKHXA

SEQ ID NO: 15 - protein, 25 aa, Unknown
  polypeptide corresponding to a section of PrP27-30
GGQGGGTHGQWNKPAKPKTAMKHXA

SEQ ID NO: 16 - protein, 24 aa, Unknown
  polypeptide corresponding to a section of PrP27-30
GQGGGTHGQWNKPAKPKTAMKHXA

SEQ ID NO: 17 - protein, 28 aa, Unknown
  polypeptide corresponding to a section of PrP27-30
GGGWGQGGGTHGQWNKPAKPKTAMKXAG

SEQ ID NO: 18 - protein, 27 aa, Unknown
  polypeptide corresponding to a section of PrP27-30
GGWGQGGGTHGQWNKPAKPKTAMKXAG

SEQ ID NO: 19 - protein, 26 aa, Unknown
  polypeptide corresponding to a section of PrP27-30
GWGQGGGTHGQWNKPAKPKTAMKXAG

SEQ ID NO: 20 - protein, 25 aa, Unknown
  polypeptide corresponding to a section of PrP27-30
WGQGGGTHGQWNKPAKPKTAMKXAG

SEQ ID NO: 21 - protein, 27 aa, Unknown
  polypeptide corresponding to a section of PrP27-30
GGGGQGGGTHGQWNKPAKPKTAMKXAG

SEQ ID NO: 22 - protein, 26 aa, Unknown
  polypeptide corresponding to a section of PrP27-30
GGGQGGGTHGQWNKPAKPKTAMKXAG

SEQ ID NO: 23 - protein, 25 aa, Unknown
  polypeptide corresponding to a section of PrP27-30
GGQGGGTHGQWNKPAKPKTAMKXAG

SEQ ID NO: 24 - protein, 24 aa, Unknown
  polypeptide corresponding to a section of PrP27-30
GQGGGTHGQWNKPAKPKTAMKXAG

SEQ ID NO: 25 - protein, 27 aa, Unknown
  polypeptide corresponding to a section of PrP27-30
GGGWGQGGGTHGQWNKPAKPKTAMKHX

SEQ ID NO: 26 - protein, 26 aa, Unknown
  polypeptide corresponding to a section of PrP27-30
GGWGQGGGTHGQWNKPAKPKTAMKHX

SEQ ID NO: 27 - protein, 25 aa, Unknown
  polypeptide corresponding to a section of PrP27-30
GWGQGGGTHGQWNKPAKPKTAMKHX

SEQ ID NO: 28 - protein, 24 aa, Unknown
  polypeptide corresponding to a section of PrP27-30
WGQGGGTHGQWNKPAKPKTAMKHX

SEQ ID NO: 29 - protein, 28 aa, Unknown
  polypeptide corresponding to a section of PrP27-30
GGGGQGGGTHGQWNKPAKPKTAMKHXAG

SEQ ID NO: 30 - protein, 25 aa, Unknown
  polypeptide corresponding to a section of PrP27-30
GGGQGGGTHGQWNKPAKPKTAMKHX

SEQ ID NO: 31 - protein, 24 aa, Unknown
  polypeptide corresponding to a section of PrP27-30
GGQGGGTHGQWNKPAKPKTAMKHX

SEQ ID NO: 32 - protein, 23 aa, Unknown
  polypeptide corresponding to a section of PrP27-30
GQGGGTHGQWNKPAKPKTAMKHX

SEQ ID NO: 33 - protein, 27 aa, Unknown
  polypeptide corresponding to a section of PrP27-30
GGGWGQGGGTHGQWNKPAKPKTAMKXA

SEQ ID NO: 34 - protein, 26 aa, Unknown
  polypeptide corresponding to a section of PrP27-30
GGWGQGGGTHGQWNKPAKPKTAMKXA

SEQ ID NO: 35 - protein, 25 aa, Unknown
  polypeptide corresponding to a section of PrP27-30
GWGQGGGTHGQWNKPAKPKTAMKXA

SEQ ID NO: 36 - protein, 24 aa, Unknown
  polypeptide corresponding to a section of PrP27-30
WGQGGGTHGQWNKPAKPKTAMKXA

SEQ ID NO: 37 - protein, 26 aa, Unknown
  polypeptide corresponding to a section of PrP27-30
GGGGQGGGTHGQWNKPAKPKTAMKXA

SEQ ID NO: 38 - protein, 25 aa, Unknown
  polypeptide corresponding to a section of PrP27-30
GGGQGGGTHGQWNKPAKPKTAMKXA

SEQ ID NO: 39 - protein, 24 aa, Unknown
  polypeptide corresponding to a section of PrP27-30
GGQGGGTHGQWNKPAKPKTAMKXA

SEQ ID NO: 40 - protein, 23 aa, Unknown
  polypeptide corresponding to a section of PrP27-30
GQGGGTHGQWNKPAKPKTAMKXA

SEQ ID NO: 41 - protein, 27 aa, Unknown
  polypeptide corresponding to a section of PrP27-30
GGGWGQGGGTHGQWNKPAKPKTAMKXG

SEQ ID NO: 42 - protein, 26 aa, Unknown
  polypeptide corresponding to a section of PrP27-30
GGWGQGGGTHGQWNKPAKPKTAMKXG

SEQ ID NO: 43 - protein, 25 aa, Unknown
  polypeptide corresponding to a section of PrP27-30
GWGQGGGTHGQWNKPAKPKTAMKXG

SEQ ID NO: 44 - protein, 24 aa, Unknown
  polypeptide corresponding to a section of PrP27-30
WGQGGGTHGQWNKPAKPKTAMKXG

SEQ ID NO: 45 - protein, 26 aa, Unknown
  polypeptide corresponding to a section of PrP27-30
GGGGQGGGTHGQWNKPAKPKTAMKXG

SEQ ID NO: 46 - protein, 25 aa, Unknown
  polypeptide corresponding to a section of PrP27-30
GGGQGGGTHGQWNKPAKPKTAMKXG

SEQ ID NO: 47 - protein, 24 aa, Unknown
  polypeptide corresponding to a section of PrP27-30
GGQGGGTHGQWNKPAKPKTAMKXG

SEQ ID NO: 48 - protein, 23 aa, Unknown
  polypeptide corresponding to a section of PrP27-30
GQGGGTHGQWNKPAKPKTAMKXG

SEQ ID NO: 49 - protein, 28 aa, Unknown
  polypeptide corresponding to a section of PrP27-30
GGGWGQGGGTHGQWNKPAKPKTAMKHXG

SEQ ID NO: 50 - protein, 27 aa, Unknown
  polypeptide corresponding to a section of PrP27-30
GGWGQGGGTHGQWNKPAKPKTAMKHXG

SEQ ID NO: 51 - protein, 26 aa, Unknown
  polypeptide corresponding to a section of PrP27-30
GWGQGGGTHGQWNKPAKPKTAMKHXG

SEQ ID NO: 52 - protein, 25 aa, Unknown
  polypeptide corresponding to a section of PrP27-30
WGQGGGTHGQWNKPAKPKTAMKHXG

SEQ ID NO: 53 - protein, 27 aa, Unknown
  polypeptide corresponding to a section of PrP27-30
GGGGQGGGTHGQWNKPAKPKTAMKHXG

SEQ ID NO: 54 - protein, 26 aa, Unknown
  polypeptide corresponding to a section of PrP27-30
GGGQGGGTHGQWNKPAKPKTAMKHXG

SEQ ID NO: 55 - protein, 25 aa, Unknown
  polypeptide corresponding to a section of PrP27-30
GGQGGGTHGQWNKPAKPKTAMKHXG

SEQ ID NO: 56 - protein, 24 aa, Unknown
  polypeptide corresponding to a section of PrP27-30
GQGGGTHGQWNKPAKPKTAMKHXG

SEQ ID NO: 57 - protein, 26 aa, Unknown
  polypeptide corresponding to a section of PrP27-30
GGGWGQGGGTHGQWNKPAKPKTAMKX

SEQ ID NO: 58 - protein, 25 aa, Unknown
  polypeptide corresponding to a section of PrP27-30
GGWGQGGGTHGQWNKPAKPKTAMKX

SEQ ID NO: 59 - protein, 24 aa, Unknown
  polypeptide corresponding to a section of PrP27-30
GWGQGGGTHGQWNKPAKPKTAMKX

SEQ ID NO: 60 - protein, 23 aa, Unknown
  polypeptide corresponding to a section of PrP27-30
WGQGGGTHGQWNKPAKPKTAMKX

SEQ ID NO: 61 - protein, 25 aa, Unknown
  polypeptide corresponding to a section of PrP27-30
GGGGQGGGTHGQWNKPAKPKTAMKX

SEQ ID NO: 62 - protein, 24 aa, Unknown
  polypeptide corresponding to a section of PrP27-30
GGGQGGGTHGQWNKPAKPKTAMKX

SEQ ID NO: 63 - protein, 23 aa, Unknown
  polypeptide corresponding to a section of PrP27-30
GGQGGGTHGQWNKPAKPKTAMKX

SEQ ID NO: 64 - protein, 22 aa, Unknown
  polypeptide corresponding to a section of PrP27-30
GQGGGTHGQWNKPAKPKTAMKX

SEQ ID NO: 65 - protein, 28 aa, Unknown
  polypeptide corresponding to a section of PrP27-30
GGGWGQGGGTHGQWNKPAKPKTAMKHAG

SEQ ID NO: 66 - protein, 27 aa, Unknown
  polypeptide corresponding to a section of PrP27-30
GGWGQGGGTHGQWNKPAKPKTAMKHAG

SEQ ID NO: 67 - protein, 26 aa, Unknown
  polypeptide corresponding to a section of PrP27-30
GWGQGGGTHGQWNKPAKPKTAMKHAG

SEQ ID NO: 68 - protein, 25 aa, Unknown
  polypeptide corresponding to a section of PrP27-30
WGQGGGTHGQWNKPAKPKTAMKHAG

SEQ ID NO: 69 - protein, 27 aa, Unknown
  polypeptide corresponding to a section of PrP27-30
GGGGQGGGTHGQWNKPAKPKTAMKHAG

SEQ ID NO: 70 - protein, 26 aa, Unknown
  polypeptide corresponding to a section of PrP27-30
GGGQGGGTHGQWNKPAKPKTAMKHAG

SEQ ID NO: 71 - protein, 25 aa, Unknown
  polypeptide corresponding to a section of PrP27-30
GGQGGGTHGQWNKPAKPKTAMKHAG

SEQ ID NO: 72 - protein, 24 aa, Unknown
  polypeptide corresponding to a section of PrP27-30
GQGGGTHGQWNKPAKPKTAMKHAG

SEQ ID NO: 73 - protein, 27 aa, Unknown
  polypeptide corresponding to a section of PrP27-30
GGGWGQGGGTHGQWNKPAKPKTAMKHA

SEQ ID NO: 74 - protein, 26 aa, Unknown
  polypeptide corresponding to a section of PrP27-30
GGWGQGGGTHGQWNKPAKPKTAMKHA

SEQ ID NO: 75 - protein, 25 aa, Unknown
  polypeptide corresponding to a section of PrP27-30
GWGQGGGTHGQWNKPAKPKTAMKHA

SEQ ID NO: 76 - protein, 24 aa, Unknown
  polypeptide corresponding to a section of PrP27-30
WGQGGGTHGQWNKPAKPKTAMKHA

SEQ ID NO: 77 - protein, 26 aa, Unknown
  polypeptide corresponding to a section of PrP27-30
GGGGQGGGTHGQWNKPAKPKTAMKHA

SEQ ID NO: 78 - protein, 25 aa, Unknown
  polypeptide corresponding to a section of PrP27-30
GGGQGGGTHGQWNKPAKPKTAMKHA

SEQ ID NO: 79 - protein, 24 aa, Unknown
  polypeptide corresponding to a section of PrP27-30
GGQGGGTHGQWNKPAKPKTAMKHA

SEQ ID NO: 80 - protein, 23 aa, Unknown
  polypeptide corresponding to a section of PrP27-30
GQGGGTHGQWNKPAKPKTAMKHA

SEQ ID NO: 81 - protein, 27 aa, Unknown
  polypeptide corresponding to a section of PrP27-30
GGGWGQGGGTHGQWNKPAKPKTAMKAG

SEQ ID NO: 82 - protein, 26 aa, Unknown
  polypeptide corresponding to a section of PrP27-30
GGWGQGGGTHGQWNKPAKPKTAMKAG

SEQ ID NO: 83 - protein, 25 aa, Unknown
  polypeptide corresponding to a section of PrP27-30
GWGQGGGTHGQWNKPAKPKTAMKAG

SEQ ID NO: 84 - protein, 24 aa, Unknown
  polypeptide corresponding to a section of PrP27-30
WGQGGGTHGQWNKPAKPKTAMKAG

SEQ ID NO: 85 - protein, 26 aa, Unknown
  polypeptide corresponding to a section of PrP27-30
GGGGQGGGTHGQWNKPAKPKTAMKAG

SEQ ID NO: 86 - protein, 25 aa, Unknown
  polypeptide corresponding to a section of PrP27-30
GGGQGGGTHGQWNKPAKPKTAMKAG

SEQ ID NO: 87 - protein, 24 aa, Unknown
  polypeptide corresponding to a section of PrP27-30
GGQGGGTHGQWNKPAKPKTAMKAG

SEQ ID NO: 88 - protein, 23 aa, Unknown
  polypeptide corresponding to a section of PrP27-30
GQGGGTHGQWNKPAKPKTAMKAG

SEQ ID NO: 89 - protein, 27 aa, Unknown
  polypeptide corresponding to a section of PrP27-30
GGGWGQGGGTHGQWNKPAKPKTAMKHG

SEQ ID NO: 90 - protein, 26 aa, Unknown
  polypeptide corresponding to a section of PrP27-30
GGWGQGGGTHGQWNKPAKPKTAMKHG

SEQ ID NO: 91 - protein, 25 aa, Unknown
  polypeptide corresponding to a section of PrP27-30
GWGQGGGTHGQWNKPAKPKTAMKHG

SEQ ID NO: 92 - protein, 24 aa, Unknown
  polypeptide corresponding to a section of PrP27-30
WGQGGGTHGQWNKPAKPKTAMKHG

SEQ ID NO: 93 - protein, 26 aa, Unknown
  polypeptide corresponding to a section of PrP27-30
GGGGQGGGTHGQWNKPAKPKTAMKHG

SEQ ID NO: 94 - protein, 25 aa, Unknown
  polypeptide corresponding to a section of PrP27-30
GGGQGGGTHGQWNKPAKPKTAMKHG

SEQ ID NO: 95 - protein, 24 aa, Unknown
  polypeptide corresponding to a section of PrP27-30
GGQGGGTHGQWNKPAKPKTAMKHG

SEQ ID NO: 96 - protein, 23 aa, Unknown
  polypeptide corresponding to a section of PrP27-30
GQGGGTHGQWNKPAKPKTAMKHG

SEQ ID NO: 97 - protein, 26 aa, Unknown
  polypeptide corresponding to a section of PrP27-30
GGGWGQGGGTHGQWNKPAKPKTAMKA

SEQ ID NO: 98 - protein, 25 aa, Unknown
  polypeptide corresponding to a section of PrP27-30
GGWGQGGGTHGQWNKPAKPKTAMKA

SEQ ID NO: 99 - protein, 24 aa, Unknown
  polypeptide corresponding to a section of PrP27-30
GWGQGGGTHGQWNKPAKPKTAMKA

SEQ ID NO: 100 - protein, 23 aa, Unknown
  polypeptide corresponding to a section of PrP27-30
WGQGGGTHGQWNKPAKPKTAMKA

SEQ ID NO: 101 - protein, 25 aa, Unknown
  polypeptide corresponding to a section of PrP27-30
GGGGQGGGTHGQWNKPAKPKTAMKA

SEQ ID NO: 102 - protein, 24 aa, Unknown
  polypeptide corresponding to a section of PrP27-30
GGGQGGGTHGQWNKPAKPKTAMKA

SEQ ID NO: 103 - protein, 23 aa, Unknown
  polypeptide corresponding to a section of PrP27-30
GGQGGGTHGQWNKPAKPKTAMKA

SEQ ID NO: 104 - protein, 22 aa, Unknown
  polypeptide corresponding to a section of PrP27-30
GQGGGTHGQWNKPAKPKTAMKA

SEQ ID NO: 105 - protein, 26 aa, Unknown
  polypeptide corresponding to a section of PrP27-30
GGGWGQGGGTHGQWNKPAKPKTAMKH

SEQ ID NO: 106 - protein, 25 aa, Unknown
  polypeptide corresponding to a section of PrP27-30
GGWGQGGGTHGQWNKPAKPKTAMKH

SEQ ID NO: 107 - protein, 24 aa, Unknown
  polypeptide corresponding to a section of PrP27-30
GWGQGGGTHGQWNKPAKPKTAMKH

SEQ ID NO: 108 - protein, 23 aa, Unknown
  polypeptide corresponding to a section of PrP27-30
WGQGGGTHGQWNKPAKPKTAMKH

SEQ ID NO: 109 - protein, 25 aa, Unknown
  polypeptide corresponding to a section of PrP27-30
GGGGQGGGTHGQWNKPAKPKTAMKH

SEQ ID NO: 110 - protein, 24 aa, Unknown
  polypeptide corresponding to a section of PrP27-30
GGGQGGGTHGQWNKPAKPKTAMKH

SEQ ID NO: 111 - protein, 23 aa, Unknown
  polypeptide corresponding to a section of PrP27-30
GGQGGGTHGQWNKPAKPKTAMKH

SEQ ID NO: 112 - protein, 22 aa, Unknown
  polypeptide corresponding to a section of PrP27-30
GQGGGTHGQWNKPAKPKTAMKH

SEQ ID NO: 113 - protein, 26 aa, Unknown
  polypeptide corresponding to a section of PrP27-30
GGGWGQGGGTHGQWNKPAKPKTAMKG

SEQ ID NO: 114 - protein, 25 aa, Unknown
  polypeptide corresponding to a section of PrP27-30
GGWGQGGGTHGQWNKPAKPKTAMKG

SEQ ID NO: 115 - protein, 24 aa, Unknown
  polypeptide corresponding to a section of PrP27-30
GWGQGGGTHGQWNKPAKPKTAMKG

SEQ ID NO: 116 - protein, 23 aa, Unknown
  polypeptide corresponding to a section of PrP27-30
WGQGGGTHGQWNKPAKPKTAMKG

SEQ ID NO: 117 - protein, 25 aa, Unknown
  polypeptide corresponding to a section of PrP27-30
GGGGQGGGTHGQWNKPAKPKTAMKG

SEQ ID NO: 118 - protein, 24 aa, Unknown
  polypeptide corresponding to a section of PrP27-30
GGGQGGGTHGQWNKPAKPKTAMKG

SEQ ID NO: 119 - protein, 23 aa, Unknown
  polypeptide corresponding to a section of PrP27-30
GGQGGGTHGQWNKPAKPKTAMKG

SEQ ID NO: 120 - protein, 22 aa, Unknown
  polypeptide corresponding to a section of PrP27-30
GQGGGTHGQWNKPAKPKTAMKG

SEQ ID NO: 121 - protein, 22 aa, Unknown
  polypeptide corresponding to a section of PrP27-30
GGQGGGTHGQWNKPAKPKTAMK

SEQ ID NO: 122 - protein, 21 aa, Unknown
  polypeptide corresponding to a section of PrP27-30
GQGGGTHGQWNKPAKPKTAMK

SEQ ID NO: 123 - protein, 25 aa, Unknown
  polypeptide corresponding to a section of PrP27-30
GGGWGQGGGTHGQWNKPAKPKTAMK

SEQ ID NO: 124 - protein, 24 aa, Unknown
  polypeptide corresponding to a section of PrP27-30
GGWGQGGGTHGQWNKPAKPKTAMK

SEQ ID NO: 125 - protein, 23 aa, Unknown
  polypeptide corresponding to a section of PrP27-30
GWGQGGGTHGQWNKPAKPKTAMK

SEQ ID NO: 126 - protein, 22 aa, Unknown
  polypeptide corresponding to a section of PrP27-30
WGQGGGTHGQWNKPAKPKTAMK

SEQ ID NO: 127 - protein, 24 aa, Unknown
  polypeptide corresponding to a section of PrP27-30
GGGGQGGGTHGQWNKPAKPKTAMK

SEQ ID NO: 128 - protein, 23 aa, Unknown
  polypeptide corresponding to a section of PrP27-30
GGGQGGGTHGQWNKPAKPKTAMK

SEQ ID NO: 129 - protein, 28 aa, Unknown
  polypeptide corresponding to a section of PrP27-30
GGGWGQGGTHGQWNKPAKPKTAMKHXAG

SEQ ID NO: 130 - protein, 27 aa, Unknown
  polypeptide corresponding to a section of PrP27-30
GGWGQGGTHGQWNKPAKPKTAMKHXAG

SEQ ID NO: 131 - protein, 26 aa, Unknown
  polypeptide corresponding to a section of PrP27-30
GWGQGGTHGQWNKPAKPKTAMKHXAG

SEQ ID NO: 132 - protein, 25 aa, Unknown
  polypeptide corresponding to a section of PrP27-30
WGQGGTHGQWNKPAKPKTAMKHXAG

SEQ ID NO: 133 - protein, 27 aa, Unknown
  polypeptide corresponding to a section of PrP27-30
GGGGQGGTHGQWNKPAKPKTAMKHXAG

SEQ ID NO: 134 - protein, 26 aa, Unknown
  polypeptide corresponding to a section of PrP27-30
GGGQGGTHGQWNKPAKPKTAMKHXAG

SEQ ID NO: 135 - protein, 25 aa, Unknown
  polypeptide corresponding to a section of PrP27-30
GGQGGTHGQWNKPAKPKTAMKHXAG

SEQ ID NO: 136 - protein, 24 aa, Unknown
  polypeptide corresponding to a section of PrP27-30
GQGGTHGQWNKPAKPKTAMKHXAG

SEQ ID NO: 137 - protein, 27 aa, Unknown
  polypeptide corresponding to a section of PrP27-30
GGGWGQGGTHGQWNKPAKPKTAMKHXA

SEQ ID NO: 138 - protein, 26 aa, Unknown
  polypeptide corresponding to a section of PrP27-30
GGWGQGGTHGQWNKPAKPKTAMKHXA

SEQ ID NO: 139 - protein, 25 aa, Unknown
  polypeptide corresponding to a section of PrP27-30
GWGQGGTHGQWNKPAKPKTAMKHXA

SEQ ID NO: 140 - protein, 24 aa, Unknown
  polypeptide corresponding to a section of PrP27-30
WGQGGTHGQWNKPAKPKTAMKHXA

SEQ ID NO: 141 - protein, 26 aa, Unknown
  polypeptide corresponding to a section of PrP27-30
GGGGQGGTHGQWNKPAKPKTAMKHXA

SEQ ID NO: 142 - protein, 25 aa, Unknown
  polypeptide corresponding to a section of PrP27-30
GGGQGGTHGQWNKPAKPKTAMKHXA

SEQ ID NO: 143 - protein, 24 aa, Unknown
  polypeptide corresponding to a section of PrP27-30
GGQGGTHGQWNKPAKPKTAMKHXA

SEQ ID NO: 144 - protein, 23 aa, Unknown
  polypeptide corresponding to a section of PrP27-30
GQGGTHGQWNKPAKPKTAMKHXA

SEQ ID NO: 145 - protein, 27 aa, Unknown
  polypeptide corresponding to a section of PrP27-30
GGGWGQGGTHGQWNKPAKPKTAMKXAG

SEQ ID NO: 146 - protein, 27 aa, Unknown
  polypeptide corresponding to a section of PrP27-30
GGWGQGGGTHGQWNKPAKPKTAMKXAG

SEQ ID NO: 147 - protein, 25 aa, Unknown
  polypeptide corresponding to a section of PrP27-30
GWGQGGTHGQWNKPAKPKTAMKXAG

SEQ ID NO: 148 - protein, 24 aa, Unknown
  polypeptide corresponding to a section of PrP27-30
WGQGGTHGQWNKPAKPKTAMKXAG

SEQ ID NO: 149 - protein, 26 aa, Unknown
  polypeptide corresponding to a section of PrP27-30
GGGGQGGTHGQWNKPAKPKTAMKXAG

SEQ ID NO: 150 - protein, 25 aa, Unknown
  polypeptide corresponding to a section of PrP27-30
GGGQGGTHGQWNKPAKPKTAMKXAG

SEQ ID NO: 151 - protein, 24 aa, Unknown
  polypeptide corresponding to a section of PrP27-30
GGQGGTHGQWNKPAKPKTAMKXAG

SEQ ID NO: 152 - protein, 23 aa, Unknown
  polypeptide corresponding to a section of PrP27-30
GQGGTHGQWNKPAKPKTAMKXAG

SEQ ID NO: 153 - protein, 26 aa, Unknown
  polypeptide corresponding to a section of PrP27-30
GGGWGQGGTHGQWNKPAKPKTAMKHX

SEQ ID NO: 154 - protein, 25 aa, Unknown
  polypeptide corresponding to a section of PrP27-30
GGWGQGGTHGQWNKPAKPKTAMKHX

SEQ ID NO: 155 - protein, 24 aa, Unknown
  polypeptide corresponding to a section of PrP27-30
GWGQGGTHGQWNKPAKPKTAMKHX

SEQ ID NO: 156 - protein, 23 aa, Unknown
  polypeptide corresponding to a section of PrP27-30
WGQGGTHGQWNKPAKPKTAMKHX

SEQ ID NO: 157 - protein, 27 aa, Unknown
  polypeptide corresponding to a section of PrP27-30
GGGGQGGTHGQWNKPAKPKTAMKHXAG

SEQ ID NO: 158 - protein, 24 aa, Unknown
  polypeptide corresponding to a section of PrP27-30
GGGQGGTHGQWNKPAKPKTAMKHX

SEQ ID NO: 159 - protein, 23 aa, Unknown
  polypeptide corresponding to a section of PrP27-30
GGQGGTHGQWNKPAKPKTAMKHX

SEQ ID NO: 160 - protein, 22 aa, Unknown
  polypeptide corresponding to a section of PrP27-30
GQGGTHGQWNKPAKPKTAMKHX

SEQ ID NO: 161 - protein, 26 aa, Unknown
  polypeptide corresponding to a section of PrP27-30
GGGWGQGGTHGQWNKPAKPKTAMKXA

SEQ ID NO: 162 - protein, 25 aa, Unknown
  polypeptide corresponding to a section of PrP27-30
GGWGQGGTHGQWNKPAKPKTAMKXA

SEQ ID NO: 163 - protein, 24 aa, Unknown
  polypeptide corresponding to a section of PrP27-30
GWGQGGTHGQWNKPAKPKTAMKXA

SEQ ID NO: 164 - protein, 23 aa, Unknown
  polypeptide corresponding to a section of PrP27-30
WGQGGTHGQWNKPAKPKTAMKXA

SEQ ID NO: 165 - protein, 25 aa, Unknown
  polypeptide corresponding to a section of PrP27-30
GGGGQGGTHGQWNKPAKPKTAMKXA

SEQ ID NO: 166 - protein, 24 aa, Unknown
  polypeptide corresponding to a section of PrP27-30
GGGQGGTHGQWNKPAKPKTAMKXA

SEQ ID NO: 167 - protein, 23 aa, Unknown
  polypeptide corresponding to a section of PrP27-30
GGQGGTHGQWNKPAKPKTAMKXA

SEQ ID NO: 168 - protein, 22 aa, Unknown
  polypeptide corresponding to a section of PrP27-30
GQGGTHGQWNKPAKPKTAMKXA

SEQ ID NO: 169 - protein, 26 aa, Unknown
  polypeptide corresponding to a section of PrP27-30
GGGWGQGGTHGQWNKPAKPKTAMKXG

SEQ ID NO: 170 - protein, 25 aa, Unknown
  polypeptide corresponding to a section of PrP27-30
GGWGQGGTHGQWNKPAKPKTAMKXG

SEQ ID NO: 171 - protein, 24 aa, Unknown
  polypeptide corresponding to a section of PrP27-30
GWGQGGTHGQWNKPAKPKTAMKXG

SEQ ID NO: 172 - protein, 23 aa, Unknown
  polypeptide corresponding to a section of PrP27-30
WGQGGTHGQWNKPAKPKTAMKXG

SEQ ID NO: 173 - protein, 25 aa, Unknown
  polypeptide corresponding to a section of PrP27-30
GGGGQGGTHGQWNKPAKPKTAMKXG

SEQ ID NO: 174 - protein, 24 aa, Unknown
  polypeptide corresponding to a section of PrP27-30
GGGQGGTHGQWNKPAKPKTAMKXG

SEQ ID NO: 175 - protein, 23 aa, Unknown
  polypeptide corresponding to a section of PrP27-30
GGQGGTHGQWNKPAKPKTAMKXG

SEQ ID NO: 176 - protein, 22 aa, Unknown
  polypeptide corresponding to a section of PrP27-30
GQGGTHGQWNKPAKPKTAMKXG

SEQ ID NO: 177 - protein, 27 aa, Unknown
  polypeptide corresponding to a section of PrP27-30
GGGWGQGGTHGQWNKPAKPKTAMKHXG

SEQ ID NO: 178 - protein, 26 aa, Unknown
  polypeptide corresponding to a section of PrP27-30
GGWGQGGTHGQWNKPAKPKTAMKHXG

SEQ ID NO: 179 - protein, 25 aa, Unknown
  polypeptide corresponding to a section of PrP27-30
GWGQGGTHGQWNKPAKPKTAMKHXG

SEQ ID NO: 180 - protein, 24 aa, Unknown
  polypeptide corresponding to a section of PrP27-30
WGQGGTHGQWNKPAKPKTAMKHXG

SEQ ID NO: 181 - protein, 26 aa, Unknown
  polypeptide corresponding to a section of PrP27-30
GGGGQGGTHGQWNKPAKPKTAMKHXG

SEQ ID NO: 182 - protein, 25 aa, Unknown
  polypeptide corresponding to a section of PrP27-30
GGGQGGTHGQWNKPAKPKTAMKHXG

SEQ ID NO: 183 - protein, 24 aa, Unknown
  polypeptide corresponding to a section of PrP27-30
GGQGGTHGQWNKPAKPKTAMKHXG

SEQ ID NO: 184 - protein, 23 aa, Unknown
  polypeptide corresponding to a section of PrP27-30
GQGGTHGQWNKPAKPKTAMKHXG

SEQ ID NO: 185 - protein, 25 aa, Unknown
  polypeptide corresponding to a section of PrP27-30
GGGWGQGGTHGQWNKPAKPKTAMKX

SEQ ID NO: 186 - protein, 24 aa, Unknown
  polypeptide corresponding to a section of PrP27-30
GGWGQGGTHGQWNKPAKPKTAMKX

SEQ ID NO: 187 - protein, 23 aa, Unknown
  polypeptide corresponding to a section of PrP27-30
GWGQGGTHGQWNKPAKPKTAMKX

SEQ ID NO: 188 - protein, 22 aa, Unknown
  polypeptide corresponding to a section of PrP27-30
WGQGGTHGQWNKPAKPKTAMKX

SEQ ID NO: 189 - protein, 24 aa, Unknown
  polypeptide corresponding to a section of PrP27-30
GGGGQGGTHGQWNKPAKPKTAMKX

SEQ ID NO: 190 - protein, 23 aa, Unknown
  polypeptide corresponding to a section of PrP27-30
GGGQGGTHGQWNKPAKPKTAMKX

SEQ ID NO: 191 - protein, 22 aa, Unknown
  polypeptide corresponding to a section of PrP27-30
GGQGGTHGQWNKPAKPKTAMKX

SEQ ID NO: 192 - protein, 21 aa, Unknown
  polypeptide corresponding to a section of PrP27-30
GQGGTHGQWNKPAKPKTAMKX

SEQ ID NO: 193 - protein, 27 aa, Unknown
  polypeptide corresponding to a section of PrP27-30
GGGWGQGGTHGQWNKPAKPKTAMKHAG

SEQ ID NO: 194 - protein, 26 aa, Unknown
  polypeptide corresponding to a section of PrP27-30
GGWGQGGTHGQWNKPAKPKTAMKHAG

SEQ ID NO: 195 - protein, 25 aa, Unknown
  polypeptide corresponding to a section of PrP27-30
GWGQGGTHGQWNKPAKPKTAMKHAG

SEQ ID NO: 196 - protein, 24 aa, Unknown
  polypeptide corresponding to a section of PrP27-30
WGQGGTHGQWNKPAKPKTAMKHAG

SEQ ID NO: 197 - protein, 26 aa, Unknown
  polypeptide corresponding to a section of PrP27-30
GGGGQGGTHGQWNKPAKPKTAMKHAG

SEQ ID NO: 198 - protein, 25 aa, Unknown
  polypeptide corresponding to a section of PrP27-30
GGGQGGTHGQWNKPAKPKTAMKHAG

SEQ ID NO: 199 - protein, 24 aa, Unknown
  polypeptide corresponding to a section of PrP27-30
GGQGGTHGQWNKPAKPKTAMKHAG

SEQ ID NO: 200 - protein, 23 aa, Unknown
  polypeptide corresponding to a section of PrP27-30
GQGGTHGQWNKPAKPKTAMKHAG

SEQ ID NO: 201 - protein, 26 aa, Unknown
  polypeptide corresponding to a section of PrP27-30
GGGWGQGGTHGQWNKPAKPKTAMKHA

SEQ ID NO: 202 - protein, 25 aa, Unknown
  polypeptide corresponding to a section of PrP27-30
GGWGQGGTHGQWNKPAKPKTAMKHA

SEQ ID NO: 203 - protein, 24 aa, Unknown
  polypeptide corresponding to a section of PrP27-30
GWGQGGTHGQWNKPAKPKTAMKHA

SEQ ID NO: 204 - protein, 23 aa, Unknown
  polypeptide corresponding to a section of PrP27-30
WGQGGTHGQWNKPAKPKTAMKHA

SEQ ID NO: 205 - protein, 25 aa, Unknown
  polypeptide corresponding to a section of PrP27-30
GGGGQGGTHGQWNKPAKPKTAMKHA

SEQ ID NO: 206 - protein, 24 aa, Unknown
  polypeptide corresponding to a section of PrP27-30
GGGQGGTHGQWNKPAKPKTAMKHA

SEQ ID NO: 207 - protein, 23 aa, Unknown
  polypeptide corresponding to a section of PrP27-30
GGQGGTHGQWNKPAKPKTAMKHA

SEQ ID NO: 208 - protein, 22 aa, Unknown
  polypeptide corresponding to a section of PrP27-30
GQGGTHGQWNKPAKPKTAMKHA

SEQ ID NO: 209 - protein, 26 aa, Unknown
  polypeptide corresponding to a section of PrP27-30
GGGWGQGGTHGQWNKPAKPKTAMKAG

SEQ ID NO: 210 - protein, 25 aa, Unknown
  polypeptide corresponding to a section of PrP27-30
GGWGQGGTHGQWNKPAKPKTAMKAG

SEQ ID NO: 211 - protein, 24 aa, Unknown
  polypeptide corresponding to a section of PrP27-30
GWGQGGTHGQWNKPAKPKTAMKAG

SEQ ID NO: 212 - protein, 23 aa, Unknown
  polypeptide corresponding to a section of PrP27-30
WGQGGTHGQWNKPAKPKTAMKAG

SEQ ID NO: 213 - protein, 25 aa, Unknown
  polypeptide corresponding to a section of PrP27-30
GGGGQGGTHGQWNKPAKPKTAMKAG

SEQ ID NO: 214 - protein, 24 aa, Unknown
  polypeptide corresponding to a section of PrP27-30
GGGQGGTHGQWNKPAKPKTAMKAG

SEQ ID NO: 215 - protein, 23 aa, Unknown
  polypeptide corresponding to a section of PrP27-30
GGQGGTHGQWNKPAKPKTAMKAG

SEQ ID NO: 216 - protein, 22 aa, Unknown
  polypeptide corresponding to a section of PrP27-30
GQGGTHGQWNKPAKPKTAMKAG

SEQ ID NO: 217 - protein, 26 aa, Unknown
  polypeptide corresponding to a section of PrP27-30
GGGWGQGGTHGQWNKPAKPKTAMKHG

SEQ ID NO: 218 - protein, 25 aa, Unknown
  polypeptide corresponding to a section of PrP27-30
GGWGQGGTHGQWNKPAKPKTAMKHG

SEQ ID NO: 219 - protein, 24 aa, Unknown
  polypeptide corresponding to a section of PrP27-30
GWGQGGTHGQWNKPAKPKTAMKHG

SEQ ID NO: 220 - protein, 23 aa, Unknown
  polypeptide corresponding to a section of PrP27-30
WGQGGTHGQWNKPAKPKTAMKHG

SEQ ID NO: 221 - protein, 25 aa, Unknown
  polypeptide corresponding to a section of PrP27-30
GGGGQGGTHGQWNKPAKPKTAMKHG

SEQ ID NO: 222 - protein, 24 aa, Unknown
  polypeptide corresponding to a section of PrP27-30
GGGQGGTHGQWNKPAKPKTAMKHG

SEQ ID NO: 223 - protein, 23 aa, Unknown
  polypeptide corresponding to a section of PrP27-30
GGQGGTHGQWNKPAKPKTAMKHG

SEQ ID NO: 224 - protein, 22 aa, Unknown
  polypeptide corresponding to a section of PrP27-30
GQGGTHGQWNKPAKPKTAMKHG

SEQ ID NO: 225 - protein, 25 aa, Unknown
  polypeptide corresponding to a section of PrP27-30
GGGWGQGGTHGQWNKPAKPKTAMKA

SEQ ID NO: 226 - protein, 24 aa, Unknown
  polypeptide corresponding to a section of PrP27-30
GGWGQGGTHGQWNKPAKPKTAMKA

SEQ ID NO: 227 - protein, 23 aa, Unknown
  polypeptide corresponding to a section of PrP27-30
GWGQGGTHGQWNKPAKPKTAMKA

SEQ ID NO: 228 - protein, 22 aa, Unknown
  polypeptide corresponding to a section of PrP27-30
WGQGGTHGQWNKPAKPKTAMKA

SEQ ID NO: 229 - protein, 24 aa, Unknown
  polypeptide corresponding to a section of PrP27-30
GGGGQGGTHGQWNKPAKPKTAMKA

SEQ ID NO: 230 - protein, 23 aa, Unknown
  polypeptide corresponding to a section of PrP27-30
GGGQGGTHGQWNKPAKPKTAMKA

SEQ ID NO: 231 - protein, 23 aa, Unknown
  polypeptide corresponding to a section of PrP27-30
GGQGGGTHGQWNKPAKPKTAMKA

SEQ ID NO: 232 - protein, 22 aa, Unknown
  polypeptide corresponding to a section of PrP27-30
GQGGGTHGQWNKPAKPKTAMKA

SEQ ID NO: 233 - protein, 26 aa, Unknown
  polypeptide corresponding to a section of PrP27-30
GGGWGQGGGTHGQWNKPAKPKTAMKH

SEQ ID NO: 234 - protein, 25 aa, Unknown
  polypeptide corresponding to a section of PrP27-30
GGWGQGGGTHGQWNKPAKPKTAMKH

SEQ ID NO: 235 - protein, 24 aa, Unknown
  polypeptide corresponding to a section of PrP27-30
GWGQGGGTHGQWNKPAKPKTAMKH

SEQ ID NO: 236 - protein, 23 aa, Unknown
  polypeptide corresponding to a section of PrP27-30
WGQGGGTHGQWNKPAKPKTAMKH

SEQ ID NO: 237 - protein, 25 aa, Unknown
  polypeptide corresponding to a section of PrP27-30
GGGGQGGGTHGQWNKPAKPKTAMKH

SEQ ID NO: 238 - protein, 24 aa, Unknown
  polypeptide corresponding to a section of PrP27-30
GGGQGGGTHGQWNKPAKPKTAMKH

SEQ ID NO: 239 - protein, 23 aa, Unknown
  polypeptide corresponding to a section of PrP27-30
GGQGGGTHGQWNKPAKPKTAMKH

SEQ ID NO: 240 - protein, 22 aa, Unknown
  polypeptide corresponding to a section of PrP27-30
GQGGGTHGQWNKPAKPKTAMKH

SEQ ID NO: 241 - protein, 26 aa, Unknown
  polypeptide corresponding to a section of PrP27-30
GGGWGQGGGTHGQWNKPAKPKTAMKG

SEQ ID NO: 242 - protein, 25 aa, Unknown
  polypeptide corresponding to a section of PrP27-30
GGWGQGGGTHGQWNKPAKPKTAMKG

SEQ ID NO: 243 - protein, 24 aa, Unknown
  polypeptide corresponding to a section of PrP27-30
GWGQGGGTHGQWNKPAKPKTAMKG

SEQ ID NO: 244 - protein, 23 aa, Unknown
  polypeptide corresponding to a section of PrP27-30
WGQGGGTHGQWNKPAKPKTAMKG

SEQ ID NO: 245 - protein, 25 aa, Unknown
  polypeptide corresponding to a section of PrP27-30
GGGGQGGGTHGQWNKPAKPKTAMKG

SEQ ID NO: 246 - protein, 24 aa, Unknown
  polypeptide corresponding to a section of PrP27-30
GGGQGGGTHGQWNKPAKPKTAMKG

SEQ ID NO: 247 - protein, 23 aa, Unknown
  polypeptide corresponding to a section of PrP27-30
GGQGGGTHGQWNKPAKPKTAMKG

SEQ ID NO: 248 - protein, 22 aa, Unknown
  polypeptide corresponding to a section of PrP27-30
GQGGGTHGQWNKPAKPKTAMKG

SEQ ID NO: 249 - protein, 22 aa, Unknown
  polypeptide corresponding to a section of PrP27-30
GGQGGGTHGQWNKPAKPKTAMK

SEQ ID NO: 250 - protein, 21 aa, Unknown
  polypeptide corresponding to a section of PrP27-30
GQGGGTHGQWNKPAKPKTAMK

SEQ ID NO: 251 - protein, 25 aa, Unknown
  polypeptide corresponding to a section of PrP27-30
GGGWGQGGGTHGQWNKPAKPKTAMK

SEQ ID NO: 252 - protein, 24 aa, Unknown
  polypeptide corresponding to a section of PrP27-30
GGWGQGGGTHGQWNKPAKPKTAMK

SEQ ID NO: 253 - protein, 23 aa, Unknown
  polypeptide corresponding to a section of PrP27-30
GWGQGGGTHGQWNKPAKPKTAMK

SEQ ID NO: 254 - protein, 22 aa, Unknown
  polypeptide corresponding to a section of PrP27-30
WGQGGGTHGQWNKPAKPKTAMK

SEQ ID NO: 255 - protein, 24 aa, Unknown
  polypeptide corresponding to a section of PrP27-30
GGGGQGGGTHGQWNKPAKPKTAMK

SEQ ID NO: 256 - protein, 23 aa, Unknown
  polypeptide corresponding to a section of PrP27-30
GGGQGGGTHGQWNKPAKPKTAMK

SEQ ID NO: 257 - protein, 23 aa, Unknown
  polypeptide corresponding to a section of PrP27-30
GQGGSHSQWNKPSKPTNMKHVGC

SEQ ID NO: 258 - protein, 31 aa, Unknown
  polypeptide corresponding to a section of PrP27-30
PGGGWNTGGSRYPGQGSPGGNRYPPQGGGXW

SEQ ID NO: 259 - protein, 30 aa, Unknown
  polypeptide corresponding to a section of PrP27-30
PGGWNTGGSRYPGQGSPGGNRYPPQGGGXW

SEQ ID NO: 260 - protein, 29 aa, Unknown
  polypeptide corresponding to a section of PrP27-30
PGWNTGGSRYPGQGSPGGNRYPPQGGGXW

SEQ ID NO: 261 - protein, 28 aa, Unknown
  polypeptide corresponding to a section of PrP27-30
GWNTGGSRYPGQGSPGGNRYPPQGGGXW

SEQ ID NO: 262 - protein, 27 aa, Unknown
  polypeptide corresponding to a section of PrP27-30
WNTGGSRYPGQGSPGGNRYPPQGGGXW

SEQ ID NO: 263 - protein, 30 aa, Unknown
  polypeptide corresponding to a section of PrP27-30
GGGWNTGGSRYPGQGSPGGNRYPPQGGGXW

SEQ ID NO: 264 - protein, 29 aa, Unknown
  polypeptide corresponding to a section of PrP27-30
GGWNTGGSRYPGQGSPGGNRYPPQGGGXW

SEQ ID NO: 265 - protein, 28 aa, Unknown
  polypeptide corresponding to a section of PrP27-30
GWNTGGSRYPGQGSPGGNRYPPQGGGXW

SEQ ID NO: 266 - protein, 30 aa, Unknown
  polypeptide corresponding to a section of PrP27-30
PGGGWNTGGSRYPGQGSPGGNRYPPQGGGX

SEQ ID NO: 267 - protein, 29 aa, Unknown
  polypeptide corresponding to a section of PrP27-30
PGGWNTGGSRYPGQGSPGGNRYPPQGGGX

SEQ ID NO: 268 - protein, 28 aa, Unknown
  polypeptide corresponding to a section of PrP27-30
PGWNTGGSRYPGQGSPGGNRYPPQGGGX

SEQ ID NO: 269 - protein, 27 aa, Unknown
  polypeptide corresponding to a section of PrP27-30
GWNTGGSRYPGQGSPGGNRYPPQGGGX

SEQ ID NO: 270 - protein, 26 aa, Unknown
  polypeptide corresponding to a section of PrP27-30
WNTGGSRYPGQGSPGGNRYPPQGGGX

SEQ ID NO: 271 - protein, 29 aa, Unknown
  polypeptide corresponding to a section of PrP27-30
GGGWNTGGSRYPGQGSPGGNRYPPQGGGX

SEQ ID NO: 272 - protein, 28 aa, Unknown
  polypeptide corresponding to a section of PrP27-30
GGWNTGGSRYPGQGSPGGNRYPPQGGGX

SEQ ID NO: 273 - protein, 27 aa, Unknown
  polypeptide corresponding to a section of PrP27-30
GWNTGGSRYPGQGSPGGNRYPPQGGGX

SEQ ID NO: 274 - protein, 30 aa, Unknown
  polypeptide corresponding to a section of PrP27-30
PGGGWNTGGSRYPGQGSPGGNRYPPQGGXW

SEQ ID NO: 275 - protein, 29 aa, Unknown
  polypeptide corresponding to a section of PrP27-30
PGGWNTGGSRYPGQGSPGGNRYPPQGGXW

SEQ ID NO: 276 - protein, 28 aa, Unknown
  polypeptide corresponding to a section of PrP27-30
PGWNTGGSRYPGQGSPGGNRYPPQGGXW

SEQ ID NO: 277 - protein, 27 aa, Unknown
  polypeptide corresponding to a section of PrP27-30
GWNTGGSRYPGQGSPGGNRYPPQGGXW

SEQ ID NO: 278 - protein, 26 aa, Unknown
  polypeptide corresponding to a section of PrP27-30
WNTGGSRYPGQGSPGGNRYPPQGGXW

SEQ ID NO: 279 - protein, 29 aa, Unknown
  polypeptide corresponding to a section of PrP27-30
GGGWNTGGSRYPGQGSPGGNRYPPQGGXW

SEQ ID NO: 280 - protein, 28 aa, Unknown
  polypeptide corresponding to a section of PrP27-30
GGWNTGGSRYPGQGSPGGNRYPPQGGXW

SEQ ID NO: 281 - protein, 27 aa, Unknown
  polypeptide corresponding to a section of PrP27-30
GWNTGGSRYPGQGSPGGNRYPPQGGXW

SEQ ID NO: 282 - protein, 29 aa, Unknown
  polypeptide corresponding to a section of PrP27-30
PGGGWNTGGSRYPGQGSPGGNRYPPQGGX

SEQ ID NO: 283 - protein, 28 aa, Unknown
  polypeptide corresponding to a section of PrP27-30
PGGWNTGGSRYPGQGSPGGNRYPPQGGX

SEQ ID NO: 284 - protein, 27 aa, Unknown
  polypeptide corresponding to a section of PrP27-30
PGWNTGGSRYPGQGSPGGNRYPPQGGX

SEQ ID NO: 285 - protein, 26 aa, Unknown
  polypeptide corresponding to a section of PrP27-30
GWNTGGSRYPGQGSPGGNRYPPQGGX

SEQ ID NO: 286 - protein, 25 aa, Unknown
  polypeptide corresponding to a section of PrP27-30
WNTGGSRYPGQGSPGGNRYPPQGGX

SEQ ID NO: 287 - protein, 28 aa, Unknown
  polypeptide corresponding to a section of PrP27-30
GGGWNTGGSRYPGQGSPGGNRYPPQGGX

SEQ ID NO: 288 - protein, 27 aa, Unknown
  polypeptide corresponding to a section of PrP27-30
GGWNTGGSRYPGQGSPGGNRYPPQGGX

SEQ ID NO: 289 - protein, 26 aa, Unknown
  polypeptide corresponding to a section of PrP27-30
GWNTGGSRYPGQGSPGGNRYPPQGGX

SEQ ID NO: 290 - protein, 29 aa, Unknown
  polypeptide corresponding to a section of PrP27-30
PGGGWNTGGSRYPGQGSPGGNRYPPQGXW

SEQ ID NO: 291 - protein, 28 aa, Unknown
  polypeptide corresponding to a section of PrP27-30
PGGWNTGGSRYPGQGSPGGNRYPPQGXW

SEQ ID NO: 292 - protein, 27 aa, Unknown
  polypeptide corresponding to a section of PrP27-30
PGWNTGGSRYPGQGSPGGNRYPPQGXW

SEQ ID NO: 293 - protein, 26 aa, Unknown
  polypeptide corresponding to a section of PrP27-30
GWNTGGSRYPGQGSPGGNRYPPQGXW

SEQ ID NO: 294 - protein, 25 aa, Unknown
  polypeptide corresponding to a section of PrP27-30
WNTGGSRYPGQGSPGGNRYPPQGXW

SEQ ID NO: 295 - protein, 28 aa, Unknown
  polypeptide corresponding to a section of PrP27-30
GGGWNTGGSRYPGQGSPGGNRYPPQGXW

SEQ ID NO: 296 - protein, 27 aa, Unknown
  polypeptide corresponding to a section of PrP27-30
GGWNTGGSRYPGQGSPGGNRYPPQGXW

SEQ ID NO: 297 - protein, 26 aa, Unknown
  polypeptide corresponding to a section of PrP27-30
GWNTGGSRYPGQGSPGGNRYPPQGXW

SEQ ID NO: 298 - protein, 28 aa, Unknown
  polypeptide corresponding to a section of PrP27-30
PGGGWNTGGSRYPGQGSPGGNRYPPQGX

SEQ ID NO: 299 - protein, 27 aa, Unknown
  polypeptide corresponding to a section of PrP27-30
PGGWNTGGSRYPGQGSPGGNRYPPQGX

SEQ ID NO: 300 - protein, 26 aa, Unknown
  polypeptide corresponding to a section of PrP27-30
PGWNTGGSRYPGQGSPGGNRYPPQGX

SEQ ID NO: 301 - protein, 25 aa, Unknown
  polypeptide corresponding to a section of PrP27-30
GWNTGGSRYPGQGSPGGNRYPPQGX

SEQ ID NO: 302 - protein, 24 aa, Unknown
  polypeptide corresponding to a section of PrP27-30
WNTGGSRYPGQGSPGGNRYPPQGX

SEQ ID NO: 303 - protein, 27 aa, Unknown
  polypeptide corresponding to a section of PrP27-30
GGGWNTGGSRYPGQGSPGGNRYPPQGX

SEQ ID NO: 304 - protein, 26 aa, Unknown
  polypeptide corresponding to a section of PrP27-30
GGWNTGGSRYPGQGSPGGNRYPPQGX

SEQ ID NO: 305 - protein, 25 aa, Unknown
  polypeptide corresponding to a section of PrP27-30
GWNTGGSRYPGQGSPGGNRYPPQGX

SEQ ID NO: 306 - protein, 30 aa, Unknown
  polypeptide corresponding to a section of PrP27-30
PGGGWNTGGSRYPGQGSPGGNRYPPQGGGW

SEQ ID NO: 307 - protein, 29 aa, Unknown
  polypeptide corresponding to a section of PrP27-30
PGGWNTGGSRYPGQGSPGGNRYPPQGGGW

SEQ ID NO: 308 - protein, 28 aa, Unknown
  polypeptide corresponding to a section of PrP27-30
PGWNTGGSRYPGQGSPGGNRYPPQGGGW

SEQ ID NO: 309 - protein, 27 aa, Unknown
  polypeptide corresponding to a section of PrP27-30
GWNTGGSRYPGQGSPGGNRYPPQGGGW

SEQ ID NO: 310 - protein, 26 aa, Unknown
  polypeptide corresponding to a section of PrP27-30
WNTGGSRYPGQGSPGGNRYPPQGGGW

SEQ ID NO: 311 - protein, 29 aa, Unknown
  polypeptide corresponding to a section of PrP27-30
GGGWNTGGSRYPGQGSPGGNRYPPQGGGW

SEQ ID NO: 312 - protein, 28 aa, Unknown
  polypeptide corresponding to a section of PrP27-30
GGWNTGGSRYPGQGSPGGNRYPPQGGGW

SEQ ID NO: 313 - protein, 27 aa, Unknown
  polypeptide corresponding to a section of PrP27-30
GWNTGGSRYPGQGSPGGNRYPPQGGGW

SEQ ID NO: 314 - protein, 29 aa, Unknown
  polypeptide corresponding to a section of PrP27-30
PGGGWNTGGSRYPGQGSPGGNRYPPQGGG

SEQ ID NO: 315 - protein, 28 aa, Unknown
  polypeptide corresponding to a section of PrP27-30
PGGWNTGGSRYPGQGSPGGNRYPPQGGG

SEQ ID NO: 316 - protein, 27 aa, Unknown
  polypeptide corresponding to a section of PrP27-30
PGWNTGGSRYPGQGSPGGNRYPPQGGG

SEQ ID NO: 317 - protein, 26 aa, Unknown
  polypeptide corresponding to a section of PrP27-30
GWNTGGSRYPGQGSPGGNRYPPQGGG

SEQ ID NO: 318 - protein, 25 aa, Unknown
  polypeptide corresponding to a section of PrP27-30
WNTGGSRYPGQGSPGGNRYPPQGGG

SEQ ID NO: 319 - protein, 28 aa, Unknown
  polypeptide corresponding to a section of PrP27-30
GGGWNTGGSRYPGQGSPGGNRYPPQGGG

SEQ ID NO: 320 - protein, 27 aa, Unknown
  polypeptide corresponding to a section of PrP27-30
GGWNTGGSRYPGQGSPGGNRYPPQGGG

SEQ ID NO: 321 - protein, 26 aa, Unknown
  polypeptide corresponding to a section of PrP27-30
GWNTGGSRYPGQGSPGGNRYPPQGGG

SEQ ID NO: 322 - protein, 29 aa, Unknown
  polypeptide corresponding to a section of PrP27-30
PGGGWNTGGSRYPGQGSPGGNRYPPQGGW

SEQ ID NO: 323 - protein, 28 aa, Unknown
  polypeptide corresponding to a section of PrP27-30
PGGWNTGGSRYPGQGSPGGNRYPPQGGW

SEQ ID NO: 324 - protein, 27 aa, Unknown
  polypeptide corresponding to a section of PrP27-30
PGWNTGGSRYPGQGSPGGNRYPPQGGW

SEQ ID NO: 325 - protein, 26 aa, Unknown
  polypeptide corresponding to a section of PrP27-30
GWNTGGSRYPGQGSPGGNRYPPQGGW

SEQ ID NO: 326 - protein, 25 aa, Unknown
  polypeptide corresponding to a section of PrP27-30
WNTGGSRYPGQGSPGGNRYPPQGGW

SEQ ID NO: 327 - protein, 28 aa, Unknown
  polypeptide corresponding to a section of PrP27-30
GGGWNTGGSRYPGQGSPGGNRYPPQGGW

SEQ ID NO: 328 - protein, 27 aa, Unknown
  polypeptide corresponding to a section of PrP27-30
GGWNTGGSRYPGQGSPGGNRYPPQGGW

SEQ ID NO: 329 - protein, 26 aa, Unknown
  polypeptide corresponding to a section of PrP27-30
GWNTGGSRYPGQGSPGGNRYPPQGGW

SEQ ID NO: 330 - protein, 28 aa, Unknown
  polypeptide corresponding to a section of PrP27-30
PGGGWNTGGSRYPGQGSPGGNRYPPQGG

SEQ ID NO: 331 - protein, 27 aa, Unknown
  polypeptide corresponding to a section of PrP27-30
PGGWNTGGSRYPGQGSPGGNRYPPQGG

SEQ ID NO: 332 - protein, 26 aa, Unknown
  polypeptide corresponding to a section of PrP27-30
PGWNTGGSRYPGQGSPGGNRYPPQGG

SEQ ID NO: 333 - protein, 25 aa, Unknown
  polypeptide corresponding to a section of PrP27-30
GWNTGGSRYPGQGSPGGNRYPPQGG

SEQ ID NO: 334 - protein, 24 aa, Unknown
  polypeptide corresponding to a section of PrP27-30
WNTGGSRYPGQGSPGGNRYPPQGG

SEQ ID NO: 335 - protein, 27 aa, Unknown
  polypeptide corresponding to a section of PrP27-30
GGGWNTGGSRYPGQGSPGGNRYPPQGG

SEQ ID NO: 336 - protein, 26 aa, Unknown
  polypeptide corresponding to a section of PrP27-30
GGWNTGGSRYPGQGSPGGNRYPPQGG

SEQ ID NO: 337 - protein, 25 aa, Unknown
  polypeptide corresponding to a section of PrP27-30
GWNTGGSRYPGQGSPGGNRYPPQGG

SEQ ID NO: 338 - protein, 28 aa, Unknown
  polypeptide corresponding to a section of PrP27-30
PGGGWNTGGSRYPGQGSPGGNRYPPQGW

SEQ ID NO: 339 - protein, 27 aa, Unknown
  polypeptide corresponding to a section of PrP27-30
PGGWNTGGSRYPGQGSPGGNRYPPQGW

SEQ ID NO: 340 - protein, 26 aa, Unknown
  polypeptide corresponding to a section of PrP27-30
PGWNTGGSRYPGQGSPGGNRYPPQGW

SEQ ID NO: 341 - protein, 25 aa, Unknown
  polypeptide corresponding to a section of PrP27-30
GWNTGGSRYPGQGSPGGNRYPPQGW

SEQ ID NO: 342 - protein, 24 aa, Unknown
  polypeptide corresponding to a section of PrP27-30
WNTGGSRYPGQGSPGGNRYPPQGW

SEQ ID NO: 343 - protein, 27 aa, Unknown
  polypeptide corresponding to a section of PrP27-30
GGGWNTGGSRYPGQGSPGGNRYPPQGW

SEQ ID NO: 344 - protein, 26 aa, Unknown
  polypeptide corresponding to a section of PrP27-30
GGWNTGGSRYPGQGSPGGNRYPPQGW

SEQ ID NO: 345 - protein, 25 aa, Unknown
  polypeptide corresponding to a section of PrP27-30
GWNTGGSRYPGQGSPGGNRYPPQGW

SEQ ID NO: 346 - protein, 27 aa, Unknown
  polypeptide corresponding to a section of PrP27-30
PGGGWNTGGSRYPGQGSPGGNRYPPQG

SEQ ID NO: 347 - protein, 26 aa, Unknown
  polypeptide corresponding to a section of PrP27-30
PGGWNTGGSRYPGQGSPGGNRYPPQG

SEQ ID NO: 348 - protein, 25 aa, Unknown
  polypeptide corresponding to a section of PrP27-30
PGWNTGGSRYPGQGSPGGNRYPPQG

SEQ ID NO: 349 - protein, 24 aa, Unknown
  polypeptide corresponding to a section of PrP27-30
GWNTGGSRYPGQGSPGGNRYPPQG

SEQ ID NO: 350 - protein, 23 aa, Unknown
  polypeptide corresponding to a section of PrP27-30
WNTGGSRYPGQGSPGGNRYPPQG

SEQ ID NO: 351 - protein, 26 aa, Unknown
  polypeptide corresponding to a section of PrP27-30
GGGWNTGGSRYPGQGSPGGNRYPPQG

SEQ ID NO: 352 - protein, 25 aa, Unknown
  polypeptide corresponding to a section of PrP27-30
GGWNTGGSRYPGQGSPGGNRYPPQG

SEQ ID NO: 353 - protein, 24 aa, Unknown
  polypeptide corresponding to a section of PrP27-30
GWNTGGSRYPGQGSPGGNRYPPQG

SEQ ID NO: 354 - protein, 28 aa, Unknown
  polypeptide corresponding to a section of PrP27-30
GGWNTGGSRYPGQGSPGGNRYPPQGGGC

(19) United States
(12) Patent Application Publication
(10) Pub. No.: US 2008/0318209 A1
(43) Pub. Date: Dec. 25, 2008

(54) Cjd Prion Testing

(75) Inventors: Matthew Richard Downham, Neuried (DE); James Francis Glover, Cheshire (GB); Rachel Emma Hanily, Suffolk (GB)

Correspondence Address: BACON & THOMAS, PLLC, 625 SLATERS LANE, FOURTH FLOOR, ALEXANDRIA, VA, 22314-1176, US

(73) Assignee: Protherics Molecular Design Ltd., Cheshire (GB)

(21) Appl. No.: 10/588,078

(22) PCT Filed: Jan. 27, 2005
(86) PCT No.: PCT/GB05/00328

§ 371 Date: Aug. 19, 2008

(30) Foreign Application Priority Data
Jan. 30, 2004 (GB) 0402123.4

(51) Int. Cl. C12Q 1/68

(52) U.S. Cl. 435/6

(57) ABSTRACT

The present invention provides an assay method for detecting infectious prion protein in a sample from a mammalian subject, said method comprising: obtaining a prion protein containing sample from said subject; contacting said sample with an agent which serves to digest non-infectious prion protein and to partially digest infected prion protein to yield a prion protein polypeptide residue; contacting the digested sample with an antibody capable of binding to a poly peptide having the amino acid sequence Vc (Gly-Gly-Gly-Trp)-Gly-Gln-Gly-Gly-R₁-R₂-His-R₃-Gln-Trp-Asn-Lys-Pro-Lys-R₄-Lys-Pro-Lys-Thr-R₅-R₆-Lys (-His-R₇-Ala-Gly) (Vc) and detecting conjugates of said antibody and said prion protein polypeptide residue; characterized in that the detection of said conjugates comprises chemical, biological or biochemical amplification of a detectable species and detection of the amplified species.

This invention relates to improvements in and relating to assay methods for testing for transmissible spongiform encephalopathy (TSE) in mammalian subjects.

Spongiform encephalopathies are a group of degenerative neurological diseases. Examples have been found in a number of mammalian species including sheep (where it is known as scrapie), cows (BSE) and humans (Creutzfeldt-Jakob disease (CJD), new variant CJD (nv CJD) and kuru). It has been reported that TSE from one species can be transmitted under laboratory conditions to mammals of another species. This crossing of species barriers by the infective agent has led to widespread concern that transfer to humans can occur as a result of ingestion of material from an infected food animal, in particular materials of bovine origin.

TSEs are characterised by a slow incubation time after which the clinical symptoms of progressive degeneration of mental state, including aggressiveness and lack of coordination, appear. Post mortems reveal a characteristic pattern of vacuolation in brain tissue due to the destruction of neural cells, and the deposition of unusual protein fibres.

Although the form of the disease found in sheep has been know for many years, spongiform encephalopathies have come to greater prominence following the appearance of BSE in cattle and of nvCJD in humans.

It is believed that the causative agent of TSE is a so-called “prion”, that is an infective agent comprising protein only and no nucleic acid. In TSE, one particular protein (termed prion protein, PrP) has been identified as the infective agent. PrP is a naturally occurring cellular protein that exists in two isoforms which differ in their tertiary structure and as a result may be distinguished by their response to enzymatic degradation, e.g. by proteinase K. Thus the non-infectious isoform niPrP is wholly digested by proteinase K while the infectious isoform iPrP is degraded to leave a detectable polypeptide residue PrP27-30.

The amino acid sequences for many mammalian PrP are known and accessible, for example on SwissProt. The amino acid sequences for the residue PrP27-30 are likewise known. There is a high degree of homology between the different mammalian PrP sequences.

Several companies have developed post mortem diagnostic tests for TSE, generally based on the use of antibodies which bind to PrP27-30 deriving from a proteolytically digested brain tissue sample.

One such assay available from Enfer, Dublin, Ireland, uses the technology described in EP-B-616613. More particularly the Enfer assay uses two polyclonal antibodies raised against immunogenic conjugates of polypeptide sequences corresponding (i) to a section of PrP27-30 and (ii) to a section of PrP outside the PrP27-30 section. These sections are referred to in EP-B-616613 as Vc and Va respectively.

While the commercial assays have met with some success there is a continuing need for improved TSE assays and in particular for assays that may be performed ante-mortem or which do not require brain tissue samples.

The present invention provides just such an improved assay.

Thus viewed from one aspect the invention provides an assay method for detecting infectious prion protein in a sample from a mammalian subject, said method comprising: obtaining a prion protein containing sample from said subject; contacting said sample with an agent which serves to digest non-infectious prion protein and to partially digest infected prion protein to yield a prion protein polypeptide residue; contacting the digested sample with an antibody capable of binding to a polypeptide having the amino acid sequence Vc

(Gly-Gly-Gly-Trp)-Gly-Gln-Gly-Gly-R₁-R₂-His-R₃-
Gln-Trp-Asn-Lys-Pro-R₄-Lys-Pro-Lys-Thr-R₅-R₆-Lys
(-His-R₇-Ala-Gly) (Vc)

(wherein R₁ is either Gly or absent;
R₂ is either Thr or Ser;
R₃ is an amino acid residue selected from Gly, Ser and Asn;
R₄ and R₅ are each independently either Asn or Ser;
R₆ is an amino acid residue selected from Met, Leu and Phe;
R₇ is either Val or Met; and wherein one or more residues within brackets may be present or absent with the proviso that if they are present they are attached to the rest of the peptide in sequence); and detecting conjugates of said antibody and said prion protein polypeptide residue; characterized in that the detection of said conjugates comprises chemical, biological or biochemical amplification, especially preferably biochemical or biological amplification, of a detectable species and detection of the amplified species.

The antibody capable of binding to Vc is preferably an antibody raised against an immunogenic conjugate of a synthetic polypeptide of amino acid sequence Vc (or more preferably Vc′ as described below), e.g. by vaccination of a mammal therewith and collection of sera or Vc-binding IgG.

The prion protein containing sample used in the method of the invention may be a sample of any body tissue, fluid or material which contains prion proteins, e.g. muscle, tonsil, brain, blood, urine, faeces, etc. The sample may also be blood, serum or plasma from blood banks, blood products (e.g. coagulation factors), tissue products, culture media containing mammalian products (e.g. BSA), pharmaceutical components derived from mammalian species (e.g. heparin), etc. For post-mortem testing, brain tissue will desirably be used due to its high prion content. For ante-mortem testing the sample is preferably a biopsy tissue sample, blood, urine or faeces. The sample is preferably pretreated to lyse any cells therein, e.g. by homogenization or other tissue disruptive methods.

The sample is then contacted with the prion protein digesting agent, e.g. proteinase K, under conditions and for a period sufficient for digestion of the non-infectious prion protein. Such treatments and treatment conditions and durations are well known in the art.

If desired the sample may be treated before and/or after digestion to separate out sample components and/or digestion products other than undigested or partially digested prion protein, e.g. by centrifugation, chromatography, etc.

Following digestion, the sample is contacted with the Vc-binding antibody. This may be prepared as described in EP-B-616613. Particularly preferably the antibody is prepared using a conjugate of a polypeptide of sequence

GQGGSHSQWNKPSKPKTNMKHVGC (Vc′)

with an immunogenic carrier, e.g. tetanus toxoid, ovalbumin, etc. Conjugation may be effected using a standard linking agent, e.g. m-maleimido-benzoyl-N-hydroxy sulphosuccinimide easter (SMBS), etc. The antibody is preferably polyclonal. Standard antibody production techniques may be used.

The Vc-binding antibody may be immobilized on a substrate (e.g. a flat surface, optionally superparamagnetic beads, rods, meshes, tubes, etc) using conventional protein immobilization techniques. Alternatively, a non-immobilized Vc-binding antibody may be used.

The precise series of steps in the detection stage of the assay method will depend on whether an immobilized or non-immobilized Vc-binding antibody is used and on the technique selected for chemical, biological or biochemical amplification.

By chemical amplification it is meant that a non-biochemical chemical reaction (e.g. a reaction catalysed by a chemical substance not normally found in a biological environment) is used to generate a detectable species, the presence or absence of which is indicative of the presence of antibody:prion protein polypeptide residue conjugates.

By biological amplification it is meant that a microorganism is used to generate a detectable species (e.g. chemical substance, microorganisms, etc), the presence or absence of which is indicative of the presence of antibody:prion protein polypeptide residue conjugates.

By biochemical amplification it is meant that a biochemical reaction (e.g. an enzymatic reaction or a nucleic acid amplification such as PCR) is used to generate the detectable species (e.g. chemical substance), the presence or absence of which is indicative of the presence of antibody:prion protein polypeptide residue conjugates.

The material which is amplified or which causes the amplification to occur may be conjugated to the Vc-binding antibody or to a further agent capable of binding to the antibody:residue conjugates, e.g. a second antibody. Where it is conjugated to the Vc-binding antibody, its ability to function may be unaffected by conjugation to the PrP27-30 or it may be activated or deactivated by such conjugation. As a result, unreacted antibody may or may not have to be separated from antibody:residue conjugates. This is conventional in immunoassay procedures and the person of ordinary skill will readily appreciate the steps that should be taken. Where the material which is to be amplified or which causes amplification is separate from the Vc-binding antibody, it will generally be necessary to separate unconjugated antibody from the antibody:residue conjugates. Again this is conventional in immunoassay procedures and the person of ordinary skill will readily appreciate the steps that should be taken.

The material which is amplified or which causes amplification may comprise more than one component. In this case, one of the components may be conjugated to the Vc-binding antibody and the other to a separate agent capable of binding to the antibody:residue conjugate. Once again it will be clear to the person of ordinary skill whether separation of unconjugated Vc-binding antibody from antibody:residue conjugates is required. Where such a two or more component system is used, it is preferably the case that the different components together create a different amplification effect from that achievable with the single components on their own, e.g. they may be catalysts (e.g. enzymes) that catalyse different stages of a multistage reaction or they may be viral agents having different effects on the same or different target microorganisms.

Where a second binding agent is used in the assay method of the invention this is preferably also an antibody. However other binding agents may be used if desired.

The term antibody as used herein, unless the content dictates otherwise, may be an antibody as such or a functional fragment (e.g. a Fab fragment) thereof, a single chain antibody or an oligomeric antibody construct. Such materials may be produced in conventional fashion.

To avoid unnecessary false negatives, the assay method of the invention also preferably involves testing a portion of the original sample for PrP content. This may be done conventionally, e.g. using PrP binding antibodies as for example in the commercial BSE tests. In this case, the PrP binding antibody should be capable of binding to iPrP and/or niPrP or fragments thereof exposed by denaturation or partial digestion. Such antibodies are described in the literature.

However in a preferred embodiment, a Va-binding antibody is used on non-PK-digested samples, i.e. analogously to the Vc-binding antibodies with the omission of the niPrP digestion step. Particularly preferably the antibody used is a Va-binding antibody as defined below. Especially preferably such an antibody is one raised against an immunogenic conjugate of a synthetic polypeptide of amino acid sequence Va (or more preferably Va′ as described below), e.g. by vaccination of a mammal therewith and collection of sera or Va-binding IgG.

By a Va-binding antibody is meant one capable of binding to a polypeptide of sequence

(Pro-Gly-Gly-R₈)-Trp-Asn-Thr-Gly-Gly-Ser-Arg-Tyr-
Pro-Gly-Gln-Gly-Ser-Pro-Gly-Gly-Asn-Arg-Tyr-Pro-
Pro-Gln-Gly-(Gly-R₉-R₁₀-Trp) (Va)

wherein R₈ and R₉ are each independently either Gly or absent;
R₁₀ is either Gly or Thr; and wherein one or more residues within brackets may be present or absent with the proviso that if they are present they are attached to the rest of the peptide in sequence.

This may be prepared as described in EP-B-616613. Particularly preferably the antibody is prepared using a conjugate of a polypeptide of sequence This may be prepared as described in EP-B-616613. Particularly preferably the antibody is prepared using a conjugate of a polypeptide of sequence

GGWNTGGSRYPGQGSPGGNRYPPQGGGC (Va′)

with an immunogenic carrier, e.g. tetanus toxoid, ovalbumin, etc. Conjugation may be effected using a standard linking agent, e.g. SMBS. The antibody is preferably polyclonal. Standard antibody production techniques may be used.

Where an ante-mortem TSE assay result is positive, it is preferable to treat the subject with a therapeutic agent comprising a Vc-binding antibody. This may be the antibody alone or may be a conjugate of the antibody with an agent which serves to hinder the transformation of niPrP to iPrP or to degrade the iPrP. In the case of human TSE, the therapeutic antibody is preferably a selected antibody (i.e. an anti-Vc or anti-Va antibody as defined above). Administration is preferably by injection or infusion, e.g. into the CSF. Dosages may be determined conventionally using TSE infected animal models. Such therapeutic use forms a further aspect of the present invention.

Viewed from a further aspect the invention provides a kit for use in the assay method of the invention, said kit comprising:
(i) a Vc-binding antibody;
(ii) optionally and preferably a Va-binding antibody;
(iii) optionally and preferably proteinase K;
(iv) a material capable of chemical, biological or biochemical amplification, preferably biological or biochemical amplification, and detection or of causing chemical, biological or biochemical amplification, preferably biological or biochemical amplification, of a detectable species, said material optionally being conjugated to antibody (i); and
(v) optionally and preferably instructions for the performance of said assay method

While the material (iv) may be an enzyme or other catalyst it is preferably a substance which is itself replicated in the amplification procedure.

The invention will now be described further with reference to the following non-binding Examples:

### EXAMPLE 1

### Sample Preparation

Initial tissue disruption using appropriate methods such as homogenisation in a detergent buffer using pestle and centrifuge tube to produce a crude homogenate which may be clarified by centrifugation. The tissue homogenate sample is diluted to a working concentration in a suitable buffer in readiness for Proteinase K digestion.

### EXAMPLE 2

### Prion Protein Digestion

To distinguish iPrP from niPrP the detection systems rely on the relative resistance of iPrP to digestion by the enzyme proteinase K (PK) (see Kretzschmar, Clin Lab Med. P 109-128 (2003)). Typical conditions of PK treatment would be, diluted homogenate sample split into two equal portions and PK added to one portion and incubated accordingly (e.g. for 30 mins at 37° C.).

### EXAMPLE 3

### Vc-Binding Antibody Addition

Serum or purified IgG are prepared from the blood of animals immunised with Vc peptide conjugate vaccine. These sera would then be diluted to a working concentration, contacted with the PK digested homogenised sample and incubated accordingly (e.g. for 1 hour at 20-25° C.).

### EXAMPLE 4

### Signal Amplification and Detection

Signal amplification would typically require applying biochemical techniques, molecular or conjugated secondary antibody techniques. One example would be the incubation of an antibody conjugated with biotin molecules (e.g. for 1 hour at 20-25° C.) amplified by the addition of Avidin (biotin binding agent) conjugated to horse radish peroxidase (e.g. for 1 hour at 20-25° C.) and detection using a chromogenic substrate, read at a specific absorbance on a spectrophotometer.

### EXAMPLE 5

### Antibody Efficacy

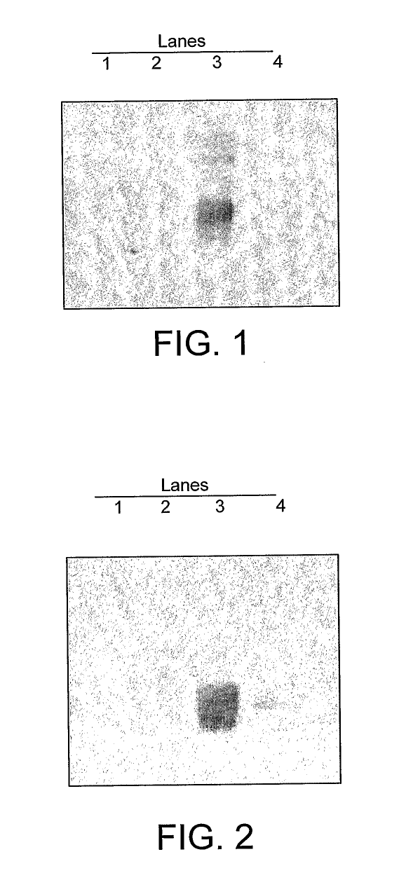

Vc-binding and Va-binding polyclonal antibodies prepared by animal immunization with antigenic conjugates of Vc′ and Va′ respectively were contacted with brain homogenate prepared as described above from CJD infected and non-infected human brain with and without proteinase K digestion. The samples were then subjected to Western blot analysis and the results are shown in accompanying FIGS. 1 and 2. FIGS. 1 and 2 are for Va-binding and Vc-binding antibodies respectively. In each Figure, lane 1 is uninfected homogenate, lane 2 is PK treated uninfected homogenate, lane 3 is infected homogenate, and lane 4 is PK-treated infected homogenate.

1. An assay method for detecting infectious prion protein in a sample from a mammalian subject, said method comprising: obtaining a prion protein containing sample from said subject; contacting said sample with an agent which serves to digest non-infectious prion protein and to partially digest infected prion protein to yield a prion protein polypeptide residue; contacting the digested sample with an antibody capable of binding to a polypeptide having the amino acid sequence
Vc (Gly-Gly-Gly-Trp)-Gly-Gln-Gly-Gly-R₁-R₂-His-R₃-Gln-Trp-Asn-Lys-Pro-Lys-R₄-Lys-Pro-Lys-Thr-R₅-R₆-Lys (-His-R₇-Ala-Gly) (Vc) SEQ. 10 1 to 256
(wherein R₁ is either Gly or absent;
R₂ is either Thr or Ser;
R₃ is an amino acid residue selected from Gly, Ser and Asn;
R₄ and R₅ are each independently either Asn or Ser;
R₆ is an amino acid residue selected from Met, Leu and Phe;
R₇ is either Val or Met; and wherein one or more residues within brackets may be present or absent with the proviso that if they are present they are attached to the rest of the peptide in sequence); and detecting conjugates of said antibody and said prion protein polypeptide residue; characterized in that the detection of said conjugates comprises chemical, biological or biochemical amplification of a detectable species and detection of the amplified species.

2. A method as claimed in claim 1 wherein said subject is human, preferably animate.

3. A method as claimed in claim 1 for detecting infectious prion protein associated with CJD, nvCJD or kuru.

4. A kit for use in the assay method of claim 1, said kit comprising:
(i) a Vc-binding antibody;
(ii) optionally a Va-binding antibody;
(iii) optionally proteinase K;
(iv) a material capable of chemical, biological or biochemical amplification and detection or of causing chemical, biological or biochemical amplification of a detectable species, said material optionally being conjugated to antibody (i); and
(v) optionally instructions for the performance of said assay method.

5. The use of a iPrP binding antibody in the manufacture of a medicament for use in the treatment of human TSE.

6. A method as claimed in claim 2 for detecting infectious prion protein associated with CJD, nvCJD or kuru.

7. A kit for use in the assay method of claim 2, said kit comprising:
(i) a Vc-binding antibody;
(ii) optionally a Va-binding antibody;
(iii) optionally proteinase K;
(iv) a material capable of chemical, biological or biochemical amplification and detection or of causing chemical, biological or biochemical amplification of a detectable species, said material optionally being conjugated to antibody (i); and
(v) optionally instructions for the performance of said assay method.

8. A kit for use in the assay method of claim 3, said kit comprising:
(i) a Vc-binding antibody;
(ii) optionally a Va-binding antibody;
(iii) optionally proteinase K;
(iv) a material capable of chemical, biological or biochemical amplification and detection or of causing chemical, biological or biochemical amplification of a detectable species, said material optionally being conjugated to antibody (i); and
(v) optionally instructions for the performance of said assay method.
